(19) United States
(12) Patent Application Publication
(10) Pub. No.: US 2022/0257429 A1
(43) Pub. Date: Aug. 18, 2022

(54) SMART SYSTEM OF DIAPER REPLACEMENT TIME ALERT WITH TEMPERATURE DETECTION SENSOR WITHOUT BODY CONTACT

(71) Applicant: Mohammad AKRAMI, Semnan (IR)

(72) Inventor: Mohammad AKRAMI, Semnan (IR)

(21) Appl. No.: 17/626,846

(22) PCT Filed: Jun. 18, 2020
(86) PCT No.: PCT/IB2020/055711

§ 371 Date: Jan. 13, 2022

(30) Foreign Application Priority Data
Jul. 22, 2019 (IR) 13985014000300344

(51) Int. Cl. A61F 13/42; A61F 13/49; A61B 5/00; A61B 5/01

(52) U.S. Cl. CPC A61F 13/42; A61F 13/49; A61B 5/6808; A61B 5/01; A61B 2562/0271; A61B 5/0024; A61B 5/0022; A61F 2013/424; A61B 5/7435

(57) ABSTRACT

This invention is a kind of alert diaper that can detect moisture and measure body temperature. The moisture detection sensor used in the invention consists of two sheet electrodes made of aluminum, which are placed on top of each other in layers and a layer of permeable and non-conductive fabric is located in the space between the electrodes. By exposing the moisture between the two electrodes, electric current is established between the two electrodes, and the current is detected by the circuit. There's also the ability to measure body temperature with a kind of infrared receiver sensor located outside the diaper. The sensor detects body temperature without contact with the body by receiving heat radiation emitted from the body. The information processed by the Bluetooth system is then displayed in application installed on mobile phone, PC and other devices equipped with the Bluetooth system.

### TECHNICAL FIELD

The technical background of the invention is in the case of replacement time alert diapers, when, exposed to urine or feces by a type of moisture sensor, announces the replacement time as an audio alarm.

### BACKGROUND

Related documents founded: U.S. Pat. No. 6,870,479B2, KR1020160077373, U.S. Pat. No. 8,975,465B2, KR1020160144273, US20180325743A1, CN106691699 and U.S. Ser. No. 10/624,796B2.

The invention of U.S. Pat. No. 6,870,479B2 consists of several sensors called life sensors and some of these sensors, including the moisture sensor, are located inside the diaper, which are connected to each other by an interface located in the diaper. The use of multiple sensors increases the weight of the diaper and reduces its softness and flexibility and, therefore, the diaper does not comply with the ergonomics principles. The type of moisture sensor used is not specified in the invention and it is said that the moisture sensor may be in the form of Fine wire mesh or several separate sensors may be placed inside the diaper. The text states that the moisture detection sensor is constantly sending a signal and the interruption of the signal means the presence of moisture in the diaper, which increases the energy consumption of the battery by the system. But in the present invention, if there is moisture, the moisture detection sensor only emits an electrical signal.

The text, describing this invention, states that diapers can be designed as disposable or reusable, also some of the sensors used in the invention which are placed inside the diaper, are not disposable and can be reused. This is not hygienically correct and may spread the infection and various diseases to other people.

Numerous sensors such as moisture sensor, gas sensor, temperature sensor and sound sensor have been used in inventions KR1020160077373. As mentioned earlier, the use of multiple sensors reduces the ergonomics of the diaper because it is necessary to design the circuit of each of these sensors in the electronic board and it will increase the weight and dimensions of the electronic board. In these inventions, urine is detected by a moisture sensor and feces by a gas sensor. But in the present invention, a sensor has been designed that can detect both urine and feces with high accuracy instead of using two separate sensors to detect urine and feces; in fact, in the present invitation urine and feces are detected in the form of a sensor.

The invention of U.S. Pat. No. 8,975,465B2 is made by placing a number of conductive wires in the diaper and the handles of the wires are connected to the transmitter kit remotely; it is said that metal sheets and strips can also be used in addition to conductive wires in the description of the invention, but in claims and technical plans conductive wires have also been mentioned. The wires inside the diaper may protrude from the surface of the diaper, causing injury or damage. In addition, due to the large number of wires or metal strips, the weight of the diaper increases and its ergonomics decreases, which it can cause discomfort to people. The invention detects urine and fecal moisture by measuring the internal electrical resistance (Impedance); The urine in the diaper may not be detected by the system in the method because the electrical resistance of the urine changes and does not have a constant value, depending on the type and amount of substances excreted by the kidneys, such as ions. For example, a drug may be taken by someone wearing a diaper, which is eventually injected into the urine through the kidneys, and the medicinal compounds in the urine may change the electrical resistance of the urine, and the diaper system may not be able to detect urine. The described text and claim No. 2 of the invention of KR1020160144273 also explains that in this invention, urine is detected based on a change in conductive wire resistance.

The moisture detection sensor consists of two or more sheet electrode bands located next to each other in the invention of US20180325743A1 and it is mentioned in the description of the invention that salt has been used to facilitate the conductivity of moisture to make it easier to detect the presence of moisture. Several temperature detection sensors have also been used in the invention located in different areas inside the diaper, which can reduce the ergonomics of the diaper. In diapers whose temperature sensor is located inside the diaper, they are discarded along with the diaper after each use of diapers or sensors which in this case it costs a lot for users.

But in the present invention, no substance has been used to increase the conductivity of moisture inside the diapers, and the electronic circuit of moisture detection is designed to detect weak currents due to low moisture, such as adult feces moisture. In addition, the temperature detection sensor located in the present invention is located outside the diaper and can be used continuously, and it detects body temperature by receiving heat waves emitted from the body to non-contact.

The moisture sensor placed in the invention of CN106691699 consists of three sheet electrodes. In the technical maps and detailed descriptions of the invention, it is clearly explained that the moisture sensor strips (FIGS. 1, 21, 22, 23 and FIGS. 5 and 6, number 5 and 6) are layered, but these strips are placed parallel to each other. According to the model of placing electrodes or conductive strips inside the diaper, it can be seen that there is no ability to detect adult feces in the invention; However, if this feature is present, due to the coexistence of conductive strips, it is possible that the moisture on the surface of the skin will collide with all the conductive strips inside the diaper and the moisture on the surface of the skin will be mistaken for urine or feces; but in the present invention by referring to “FIG. 2, Technical Maps” and “Description of the Invention”, it can be seen that the electrodes 4 and 5 which are placed inside the diaper are layered on top and bottom of each other and are matched parallel to each other and the use of the method in designing the sensor is due to the fact that, according to the high power of the electronic circuit in detecting weak currents due to low moisture, the moisture on the surface of the skin caused by sweating or other factors cannot come into contact with both electrodes, therefore it increases the accuracy of the device in detecting the moisture; And this is while the adult feces, which has more moisture than the moisture on the surface of the skin, is detected by the system.

The invention of U.S. Ser. No. 10/624,796B2 was made to detect urine using the Bioelectrogenesis method and the placement of positive and negative sheets inside the diaper.

A sheet sensor has been used in the invention to detect urine whose positive and negative electrodes are serrated and placed next to each other. One of the electrodes was made of aluminum and the other electrode is a mixture of carbon and a catalyst. In the method, electricity is generated when the electrodes contact with urine and the generated electricity is transferred to the electronic circuit whose board is located inside the diaper and is stored in the capacitors in the circuit. If the generated voltage exceeds the threshold and fills the capacitors; the alarm signal is emitted by the Bluetooth module. It has been stated, in the description of the invention, a voltage booster is used to detect a small amount of urine. According to the description given, the system is not able to detect feces, especially adult feces, and can only detect urine because the moisture in the feces is so low that the catalyst in the electrode composition cannot generate electric poles and in the case of creating positive and negative poles, the current and voltage created is so weak that they do not have the power to charge the capacitors. On the other hand, only urine detection has been discussed in the invention and fecal detection has not been discussed by this system. Also, in the invention, a temperature sensor that detects body temperature is attached to the skin and measures body temperature; however, the information obtained by measuring the body temperature is not displayed to the user by the sensor, and this feature is only used to determine whether the diaper is covered or not to prevent unrealistic alarms. In addition, sticking an object to the skin in an environment with high moisture can cause skin allergies. The electronic circuit board that processes the moisture sensor and temperature sensor information is located inside the diaper and the Bluetooth module is located outside the diaper. Inserting the electronic board inside the diaper reduces ergonomics and comfort and causes discomfort to people using the diaper. On the other hand, the board located inside the diaper with the temperature sensor is disposable and is discarded after each use which imposes a high cost on people who use the diaper, but in the present invention, the moisture in diapers is detected in a different way; in addition to detecting the urine, it can also detect the feces of infants and adults and the temperature detection sensor in the system senses body temperature without contacting the body and displays it to the user which is explained in more detail below.

On Feb. 13, 2020 a report on a smart diaper that informs about the presence of moisture was published on the MIT University news website at http://news.mit.edu/. In the diaper, an RFID tag is used to detect urine, which is installed inside and under the middle layer of the diaper, where the moisture superabsorbent powder is located. Superabsorbent powders are commonly used in most diapers, and when exposed to high moisture, they absorb moisture and increase volume. In addition, by absorbing the moisture, they have a poor conductivity and this conductivity increases the radio signal by the label on the diaper which is detected by the device. Using the method, only urine can be detected and feces cannot be detected because the moisture in the feces, especially in adults, is much less than in the urine and in some cases, the moisture does not have the power to pass through the layers of diapers and contact with the superabsorbent powder and if the moisture reaches the superabsorbent powder, its amount is not enough to create a conductive property in the mentioned powder.

Receiver of radio waves of the system is able to receive signals at a distance of about one meter from the diaper and at longer distances it is not possible to detect waves. In addition, it is not possible to place a temperature detection sensor in the system.

But in the present invention, in addition to urine, infants and adults feces are detected, and the Bluetooth system in the electronic board circuit can communicate with mobile phones and other devices at much greater distances than the diapers mentioned above.

In conclusion, all diapers can be divided into three categories. The first category is diapers that use small, hard sensors. In this category, there may not be the ability to detect feces due to the small moisture sensor used and also the lack of coverage of the entire length of the diaper by the moisture sensor; and if they can detect feces, several sensors are located in different areas of the diaper, which increases the thickness and reduces the softness and flexibility, and ultimately reduces the ergonomics of the diaper. In these diapers, the sensors may be in contact with moisture directly and can be reused, in which case it is necessary to wash and disinfect the sensors for reuse, which is not hygienically correct, and it can spread the disease to other people. However, the sensors may be disposable and discarded after each use with the diaper, which also increases the cost.

The second category is diapers that detect the moisture inside the diaper based on electrical resistance. In these systems, the error in urine detection is high and the urine may not be detected by the system because the type and amount of substances excreted by the kidneys from the body are not always constant, and it may change depending on people's diet; this change in excretion can cause a change in the electrical resistance of the urine.

The third category is diapers which use moisture detection sensors with sheet conductor strips. In these inventions, sheet sensors are located parallel to each other and cover the entire length of the diaper. However, these diapers either do not have the ability to detect feces or, if they do have the ability to detect feces, they may mistakenly detect moisture on the surface of the skin caused by sweating or other factors. In fact, when a wet body collides with the inner surface of the diaper, due to the fact that the moisture detection sensor conductor strips are side by side, the moisture on the surface of the skin contacts with the conductive strips and is mistaken for urine or feces. It should be noted that in some of these inventions, a variety of salts and substances have been used to increase the conductivity of moisture, which increases the conductivity. These systems have low accuracy in detecting moisture and do not detect feces in them because the moisture from the feces, especially in adults, is not enough to combine with the material placed in the diaper and be detected by the system.

But in the present invention, all the above problems have been solved by designing a kind of sheet moisture sensor whose electrodes are placed parallel to each other. Moreover, the electronic circuit of this system has a high accuracy in receiving the moisture sensor signals and has the ability to detect weak signals and therefore there is no need to put different materials inside diapers such as all kinds of salt to increase the conductivity of moisture.

### BRIEF DESCRIPTION OF THE DRAWINGS

FIG. 1: General view of the diaper and displaying the direct connection of the electronic board to the moisture sensor electrodes

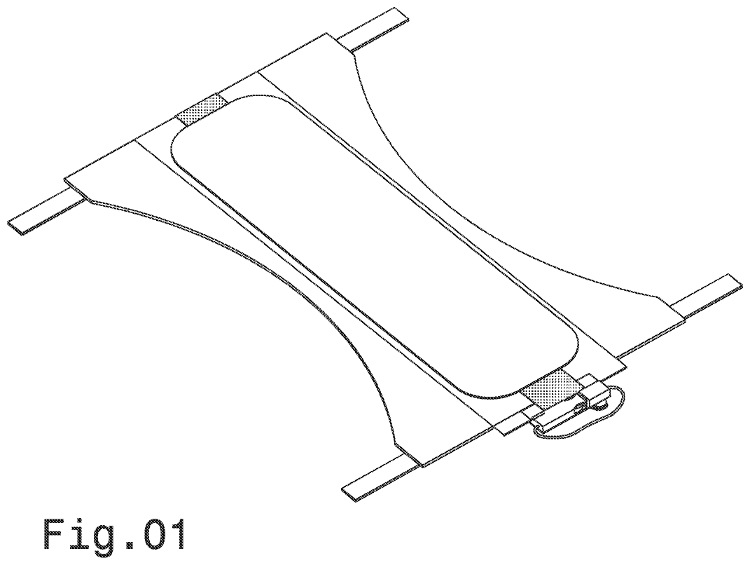

FIG. 2: Explosive view and displaying how the moisture sensor electrodes are placed inside the diaper

FIG. 3: Explosive view of electronic board and displaying how the moisture detection sensor is placed in the electronic board frame

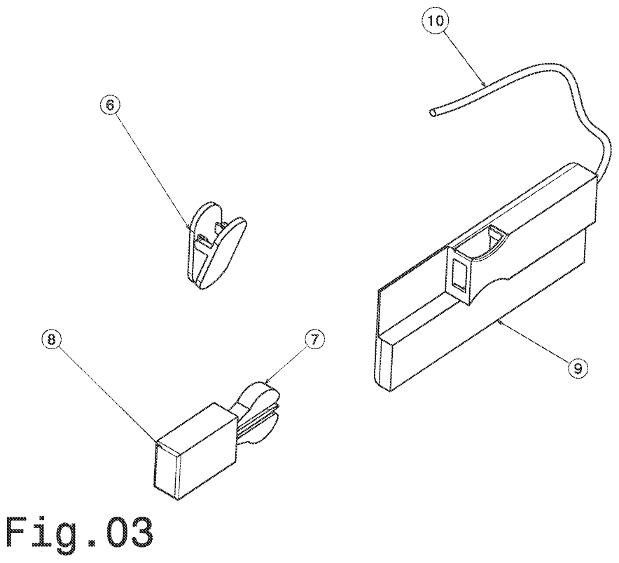

FIG. 4: A view of the electronic board on the diaper and the connection of the temperature sensor to the electronic board by wire

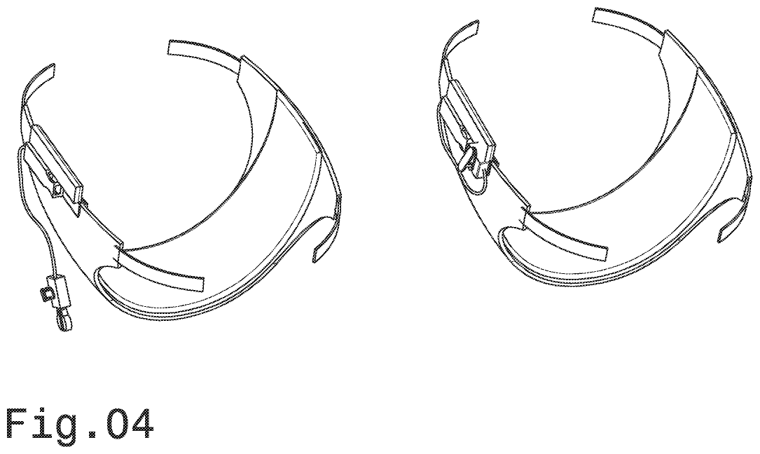

FIG. 5: A view of diapers along with electronic board and temperature detection sensor

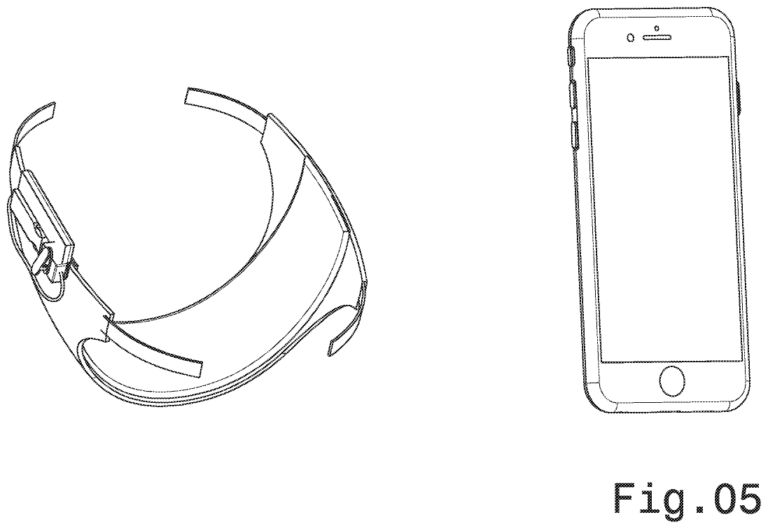

### DESCRIPTION OF THE INVENTION

The moisture detection sensor used in the invention consists of two sheet electrodes made of aluminum, and the electrodes are layered at the top and bottom. The first layer is the electrode located at the top (4) and has small and large holes and the second layer (3) is a thin, non-conductive fabric with a very high permeability that is placed between two electrodes (between the first and third layers) and the third layer (5) is the electrode that is located at the bottom. To prevent diaper permeability; the third layer is also porous and perforated, but the holes in the layer do not match the first layer. To prevent direct contact of the first layer of the sensor with the body, a layer of permeable and non-conductive fabric (2) covers the sensor when the diaper is made on the upper electrode.

To detect the moisture by the circuit, the electronic board 9 is connected to electrodes 4 and 5 and a weak electric charge is generated in the electrodes by the battery placed in the circuit, and one of the electrodes takes a negative charge and the other a positive charge. When urine or feces are placed in the diaper, the moisture from the urine and feces contacts with the first layer (4), which is the same electrode at the top, and the first layer (4) is wetted; the moisture, then, passes through the holes in the surface of the first layer and contacts with the second layer (3); because the second layer absorbs moisture, is permeable and thin, the moisture passes through it and reaches the third layer (5), which is the same electrode located at the bottom. In this way, the conductive material contact with both electrodes and the circuit is closed. As the circuit doses, electric currents flow between the anode and the cathode, and this current is detected by the circuit. To protect the environment, the battery placed in the circuit is not disposable and is charged by the Micro USB port.

Urine is highly conductive due to the presence of various ions, and infants feces is also a good conductor of electricity due to its watery nature. However, the moisture from feces of adults is low and therefore does not have high electrical conductivity, and when the moisture from the feces is placed between the electrodes, a very weak current is established between the electrodes, which it, alone, is not detectable for the circuit. To solve this problem, a transistor has been used to amplify the weak input currents, and no by-products have been used to increase the moisture conductivity inside the diaper. To better detect feces by the system, holes in the upper electrode, or the first layer (4) in the Anus area, are designed to be slightly larger than other areas.

The electronic board of the system (9) is placed outside the diaper and has the ability to be moved and used continuously, and there are no parts and devices inside the diaper except the moisture detection sensor. Diaper ergonomics are maintained and are suitable due to the sheet and flexible nature of the sensor and the circuit design of moisture detection and temperature detection systems in an electronic board.

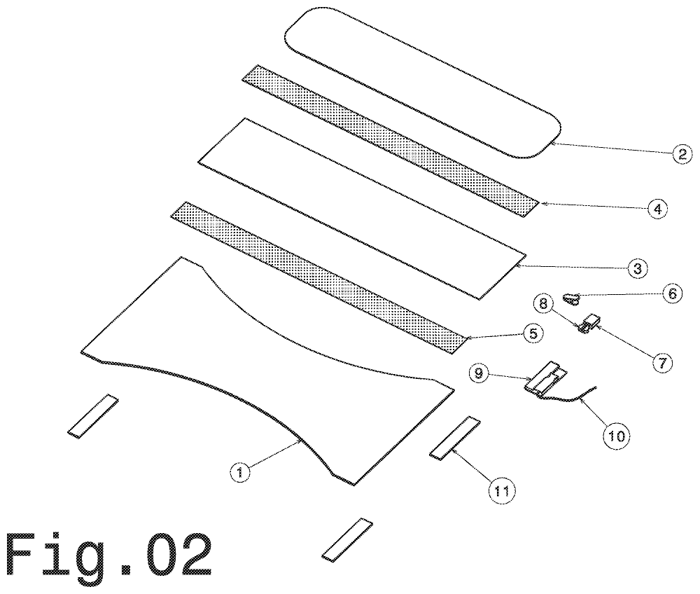

The use of layered moisture sensor in the invention is due to the high accuracy of the electronic circuit in detecting weak currents. If the electrodes were placed side by side and parallel to each other, it was possible that, when the body hits the sensor, the moisture from the sweat on the surface of the skin may be absorbed and mistakenly detected in the urine or feces. But to solve the problem in this invention, the electrodes are placed parallel to each other but up and down in the diaper (FIG. 2) and the moisture from the sweat only contact with the first layer (4) but does not pass through it; because the moisture on the surface of the skin which is less than the moisture in the urine and feces is absorbed by layer 2 which covers the electrode at the top; and because electrode 4 is made of aluminum and is resistant to moisture penetration, the moisture on the surface of the skin does not penetrate the lower layers. It should be noted that the holes placed on the surface of the electrode 4 are designed to pass more moisture such as urine and feces; and the moisture from perspiration, because it is absorbed by layer 2, does not have the power to pass through the holes in the surface of electrode 4. According to the explanation given, there are no restrictions on the use of skin creams in the diaper, and this feature is very useful in cases where it is not possible to change the diaper after the alarm is issued. The electronic board is connected directly to the sheet electrodes (4 and 5) by damps or small magnetic connectors located outside the diaper. In the invention, no wire is used to connect the sheet electrode to the electronic board.

The electronic board (9) is connected to the mobile phone, PC and other devices with Bluetooth system by Bluetooth, and when the moisture is detected inside the diaper, the Bluetooth system emits an alarm signal and parents or nurses will be notified of the presence of urine or feces in the diaper by the sound alarm generated by the application installed on the mobile phone. The application is designed to receive and store alarms for various platforms; For example, the application is installed on a mobile phone, and in addition to creating an audio alarm for changing diapers, it has the ability to automatically save alarms in order of date and time, classification of alarms, as well as displaying alarms on the chart based on date and time.

An infrared receiver sensor (8) is located outside the diaper and receives and processes the heat waves emitted by human body heat, which is the same as infrared, it detects body temperature based on the intensity of infrared radiation emitted from the body. The microcontroller for the system in the circuit is programmed in such a way that if nurses or parents activate the feature with the application, the system automatically senses the body temperature during the preset intervals and transmits it to mobile phone via Bluetooth and the body temperature is displayed in degrees Celsius, and if the temperature is above 37.5 degrees Celsius, the audio alarm will be activated.

The sensor (8), which is located outside the diaper, is connected to the electronic board (9) by the wire (10) and is placed in the Electronic board cover frame; In order to use it, the sensor is slid out of the frame and attached to the edge of the diaper or clothing by a mechanical clip (6) in such a way that it is in front of the skin. The sensor does not contact with the body directly and does not stick to the body, but in order to minimize the error in the temperature sensor, the frame in which the sensor is located (8) is designed in such a way that the sensor should be as close to the body as possible so that it can receive the heat waves emitted from the surface of the body well.

In this section, there is also the ability to automatically save the body temperature and alarm alerts for high fever based on date and time, classification of alarms as well as display them on the chart.

1. In the invention, the moisture detection sensor is layered and the electrodes of this sensor, having sheet-structure are placed parallel to each other.

2. According to claim 1, the sensor is made of two electrodes which one of these electrodes is at the top and the other at the bottom, and is located in the space between the two insulating and permeable layer electrodes; a layer of moisture-absorbing and permeable fabric (2) also covers the surface of the electrode at the top (4).

3. According to claim 1, there are many small holes on the surface of the electrodes of the sensor. These non-matching holes are designed to allow moisture to penetrate all layers of the sensor, as well as to prevent the diaper from becoming less permeable.

4. In the invention, a high-precision electronic board has been designed to detect weak electrical signals of feces and the weak signals sent by the moisture detection sensor are amplified by the transistors in the circuit.

5. According to claim 4, in the invention, no substance, including all types of salts, has been used to increase the moisture conductivity inside the diaper.

6. According to claim 2, to increase efficiency in adult feces detection, holes in the surface of electrode 4 in the Anus area are slightly larger than in other areas, and the fabric covering the electrode at the top (2) may have holes in this area that are perfectly aligned with the holes in the surface of the first electrode (4).

7. According to claim 5, there are no electronic components or devices inside the diaper other than the paper moisture detection sensor.

8. According to claim 4, the moisture on the surface of the skin, which can be caused by sweating or other factors, may be mistaken for urine or feces due to the high accuracy of the electronic circuit in receiving weak signals from the moisture detection sensor; However, due to the layered electrodes of the moisture sensor, it does not interfere with the operation of the sensor and does not cause a fake alarm.

9. According to claim 8, the moisture on the surface of the skin is absorbed by layer 2, which covers electrode 4, and reaches the electrode at the top (4). However, because the amount of the moisture is very low, it does not have the power to pass through the holes in the surface of the electrode 4, and so the moisture on the surface of the skin does not affect the performance of the sensor.

10. According to claim 9, in the invention, there are no restrictions on the production of diapers containing plant extracts as well as the use of skin creams by the user.

11. In the designed software, to display alarms, there are capabilities to classify alarms based on date and time, delete and add categories, as well as display alarms on the chart.
